(19) United States
(12) Patent Application Publication
(10) Pub. No.: US 2021/0158956 A1
(43) Pub. Date: May 27, 2021

(54) SYSTEM AND METHOD FOR INTELLIGENT ADMINISTERING OF MEDICINE

(71) Applicants: Gerard S. Rodziewicz, Fayetteville, NY (US); Dimitrios Kritikos, Moorestown, NY (US)

(72) Inventors: Gerard S. Rodziewicz, Fayetteville, NY (US); Dimitrios Kritikos, Moorestown, NY (US)

(21) Appl. No.: 16/694,530

(22) Filed: Nov. 25, 2019

(51) Int. Cl. G16H 40/67; G16H 10/60; G16H 20/17; G16H 20/90; G16H 50/70; G06N 20/00

(52) U.S. Cl. CPC G16H 40/67; G16H 10/60; G06N 20/00; G16H 20/90; G16H 50/70; G16H 20/17

(57) ABSTRACT

A system and method for intelligent administering of medicine serves to intelligently recommend an optimal dosage for administration of a medicine. An iterative machine learning algorithm learns from the behavior of a patient, through a biometric wearable monitor that collects physiological signals from sleeping and activity of the patient, and patient variables collected directly from the patient. The iterative machine learning algorithm adjusts the dosage of the medication based on different physiological signals, including: sleeping patterns, activity levels, emotional behavior, medical history, and medical needs. After receiving the recommended dosage for the medicine, the patient provides feedback, which is inputted into the iterative machine learning algorithm. The feedback allows the iterative machine learning algorithm to customize the medicine administration event to maximize the effectiveness of the medicine to the patient while reducing or even eliminating the medicine's side effects.

### FIELD OF THE INVENTION

The present invention relates generally to a system and method for intelligent administering of medicine. More so, the present invention relates to a system and method operable on a software application configured to intelligently recommend appropriate administration of medicine for the treatment of a variety of symptoms which may include but not be limited to chronic pain, anxiety, attention deficit disorders and sleep disorders, by using an iterative machine learning algorithm that learns from the behavior of a patient, through a biometric wearable monitor and patient variables collected from the patient; whereby the iterative machine learning algorithm processes and associates the data with a medicine administration event, such as the dosage of the medication based on the patient's feedback, activity levels, sleeping patterns, emotional behavior, medical history, and medical needs.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

It is known in the art that many drugs contain a chemical substance that crosses the blood-brain barrier and acts primarily upon the central nervous system where they affect brain function, providing a therapeutic utility for treatment of problems like pain, anxiety or PTSD.

Typically, software applications that use machine learning algorithms to predict medicine dosing for patients allow a patient to input data, and then use this patient-specific data to generate a medicine dosage recommendation for the patient's diagnosis. Independent variables feeding these machine learning algorithms include a library of population-based patient demographics (age, sex), along with a library of medications with their purported efficacy for various diagnoses. Some of these algorithms are iterative, i.e. a patient can access them serially to update recommendations. It is also known in the art to use calculators that input patient demographics and diagnoses, then return for example a dosing medicine that the algorithm predicts will help with this patient's problems.

Other proposals have involved calculating dosages for medications. The problem with these recommendation systems is that they do not provide intelligent machine learning that iterates to generate a recommended optimal dosage of the medicine. The prior art also does not provide a biometric wearable monitor to generate datasets about the sleeping and activity levels of the patient, so as to recommend optimal dosages and types of medicine. A system and method for intelligent administering of medicine that intelligently recommends appropriate administration of medicine through use of an iterative machine learning algorithm that learns from the behavior of a patient, through a biometric wearable monitor and patient variables collected from the patient; whereby the iterative machine learning algorithm processes and associates the data with a medicine administration event, such as the dosage of the medication based on the patient's feedback, activity levels, sleeping patterns, emotional behavior, medical history, and medical needs, is still desired.

### SUMMARY

Illustrative embodiments of the disclosure are generally directed to a system and method for intelligent administering of medicine. The system and method are operable on a software application, such as is downloadable on a mobile communication device. The system and method provide a machine learning algorithm configured to intelligently recommend appropriate administration of a medicine. The iterative machine learning algorithm learns from the behavior of a patient, through a biometric wearable monitor that collects physiological signals from sleeping and activity of the patient, and patient variables collected directly from the patient.

The iterative machine learning algorithm works to adjust the dosage of the medication based on different physiological signals, including: sleeping patterns, activity levels, emotional behavior, medical history, and medical needs. After receiving the recommended dosage for the medicine, patient provides feedback to the iterative machine learning algorithm, which allows the iterative machine learning algorithm to customize the medicine administration event more closely to the needs of the patient.

In one aspect, a computer-implemented method for intelligent administering of medicine, comprises:
attaching a biometric wearable monitor to a patient, the biometric wearable monitor being operable to record at least one physiological signal generated by the patient;
communicating, by the biometric wearable monitor, with a remote data storage device, the data storage device being operable to receive and store the physiological signal, the data storage device comprising an iterative machine learning algorithm operable to process the physiological signal;
transmitting the at least one physiological signal from the biometric wearable monitor to the remote data storage device;
collecting at least one patient variable from the patient;
transmitting the at least one patient variable to the remote data storage device;
inputting the physiological signal and patient variable into the iterative machine learning algorithm;
associating, by the iterative machine learning algorithm, the at least one physiological signal and the at least one patient variable, with at least one medicine administration event;
transmitting the medicine administration event to the patient;
repeating the transmission of the medicine administration event to a schedule; and
providing feedback from the patient for processing by the iterative machine learning algorithm.

In another aspect, the medicine comprises any medicine used to treat a particular problem such as sleep or pain.

In another aspect, the biometric wearable monitor is worn on the wrist, legs, ankle, neck, head, or torso of the patient.

In another aspect, the at least one physiological signal comprises an ECG signal and an accelerometer signal.

In another aspect, the at least one medicine administration event includes at least one of the following: a dosage of the medicine, a type of the medicine, and the schedule for administering the medicine.

In another aspect, the at least one patient variable includes at least one of the following: age, sex, current medication, current dosage of the medication, and side effects.

In another aspect, the method further comprises a step of transmitting the medicine administration event to a mobile communication device of the patient.

In another aspect, the method further comprises a step of emitting, through the biometric wearable monitor or the mobile communication device, an alert to indicate reception of the medicine administration event.

In another aspect, the mobile communication device comprises a smart phone.

In another aspect, the mobile communication device comprises a software application.

In another aspect, the method further comprises a step of recording, by the biometric wearable monitor, sleep and activity data of the patient.

In another aspect, the method further comprises a step of transmitting the medicine administration event to the patient, at least partially based on the recorded sleep and activity data.

In another aspect, the method further comprises a step of transmitting the medicine administration event to the patient in real time.

In another aspect, the method further comprises a step of transmitting a recommended dosage of the medicine administration event to the patient in real time.

In another aspect, the remote data storage device includes at least one of the following: a server, a database, a cloud-based storage system, and a processor.

One objective of the present invention is to automate the recommendation of an appropriate dosage of medicine to the patient based on the sleep and activity behavior of the patient.

Another objective is to increase the effectiveness of medicine by creating a more efficient and customizable dosage.

Yet another objective is to improve medical recommending algorithms.

An exemplary objective is to adjust a patient's dosing of medication for each of their problems to achieve maximum problem relief with minimum side effects.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a block diagram view of an exemplary system for intelligent administering of medicine, in accordance with an embodiment of the present invention;

FIG. 2 illustrates a flowchart of an exemplary method for intelligent administering of medicine, in accordance with an embodiment of the present invention;

FIG. 3 illustrates a flowchart of an alternative process for intelligent administering of medicine, in accordance with an embodiment of the present invention;

FIG. 4 illustrates an exemplary biometric wearable monitor, a mobile communication device, and a remote data storage device having an integrated machine learning algorithm, in accordance with an embodiment of the present invention; and

FIG. 5 illustrates a flowchart of an alternative method for intelligent administering of medicine, in accordance with an embodiment of the present invention.

Like reference numerals refer to like parts throughout the various views of the drawings.

### DETAILED DESCRIPTION OF THE INVENTION

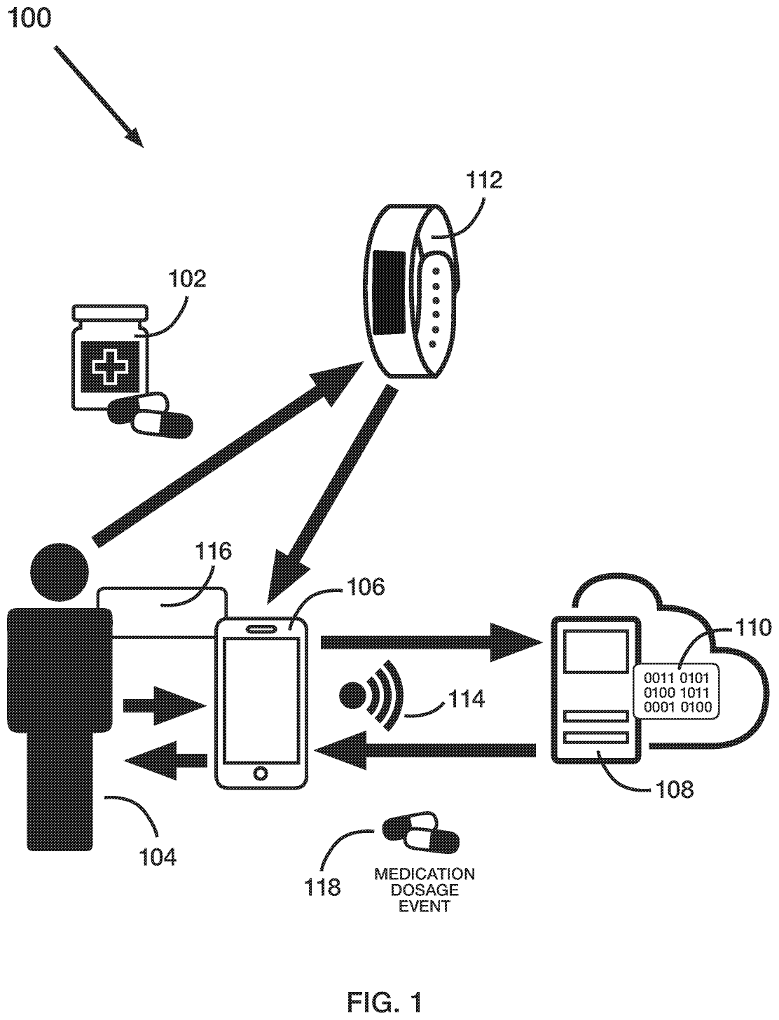

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

A system 100 and method 200 for intelligent administering of medicine 102 is referenced in FIGS. 1-5. System 100 for intelligent administering of medicine 102, hereafter “system 100”, provides an automated means to recommend the optimal dosage of medicine 102 for a patient 104. The real time medicine recommendations provided by system 100 allow the patient 104 to enhance the efficacy of the medicine 102; thereby achieving maximum problem relief with minimum side effects. Medicine 102 may include, without limitation, medications, herbs, pills, syrups, IV's, Chinese medicine, and Western medicine. It is known in the art that improper dosing of most medications may cause unwanted side effects and may be hazardous to the health; and therefore, an appropriate dosage is beneficial.

In one non-limiting embodiment shown in FIG. 1, system 100 is a computer-implemented system, operable on a software application, such as is downloadable on a mobile communication device 106. Mobile communication device 106 may include, without limitation, a smart phone, a tablet, a laptop, and a computer. System 100 is configured to intelligently recommend appropriate administration of a medicine 102 through use of an iterative machine learning algorithm 110 that learns from the behavior of a patient 104 who is wearing a biometric wearable monitor 112. Biometric wearable monitor 112 detachably attaches to the patient 104 at various body parts, including the wrist, legs, ankle, neck, head, or torso of the patient 104.

In one possible embodiment, biometric wearable monitor 112 serves to transmit two kinds of physiological signals to a software application on the patient's mobile communication device 106. A first collected physiological signal is generated by the patient 104, and can include sleep data: e.g. hours of sleep and sleep phases during the night. A second physiological signal may include steps taken by the patient 104, including number of steps taken by the patient 104 during a day. These two classes of objective data are used as independent variables that are inputted into the iterative machine learning algorithm 110.

In one non-limiting embodiment, the physiological signal 114 generated by biometric wearable monitor 112 comprises an ECG signal and an accelerometer signal. However, in other embodiments, physiological signal 114 includes data that is captured using specialized captors that transform an input signal into an electrical signal that is amplified and visualized by measurement instruments.

In some embodiments, iterative machine learning algorithm 110 is integral in a remote data storage device 108. In some embodiments, a remote data storage device 108 may include, without limitation, a server, a database, a cloud, and a processor. Remote data storage device 108 receives the physiological signal 114 recorded by the biometric wearable monitor 112, and the patient variable 116 collected from the patient 104.

In one possible embodiment, iterative machine learning algorithm 110 receives the physiological signal 114 generated by the biometric wearable monitor 112. Iterative machine learning algorithm 110 also works to adjust the dosage of the medication based on the different physiological signals, including: patient feedback, sleeping patterns, emotional behavior, medical history, and medical needs. The iterative machine learning algorithm 110 also receives at least one patient variable 116 collected from the patient 104. The patient variable 116 may include: age, sex, current medication, current dosage of the medication, side effects, allergies, and medical history. However, in other embodiments, other subjective information about the patient 104 may also be used. Patient variable 116 may be collected on a written form, an online form, or a verbal question session.

In one non-limiting embodiment, iterative machine learning algorithm 110 comprises a mathematical function operable to store and process a pre-processed dataset (physiological signals 114 and patient variable 116). The pre-processed dataset is transmitted to the remote data storage device 108, and inputted into the iterative machine learning algorithm 110. After processing and model building with the dataset, the model is tested, and then the results are matched with an expected output, such as a medicine administration event 118, i.e., the recommended dosage of medical cannabis.

For example, the sleeping patterns of the patient 104 are combined with the age of the patient 104, the types of medication used by the patient 104, and the dosing history of the patient 104 to derive a recommended dosage of the appropriate medicine over a 24-hour period. The patient 104 provides feedback through a software application, and the feedback is returned to the remote data storage device 108. The feedback is inputted into the iterative machine learning algorithm 110 to further learn and fine tune the medicine administration event 118, i.e., the recommended dosage of the appropriate medication. In some embodiments, the algorithm 110 may utilize probabilities, statistics, artificial intelligence, and other machine learning processes known in the art.

By recording physiological signals and recording patient variable 116 directly from the patient 104, the software application can utilize both subjective and objective feedback from the patient 104 as input for the iterative machine learning algorithm 110. The iterative machine learning algorithm 110 processes this data, and then outputs at least one medicine administration event 118 for the patient 104.

In some embodiments, the medicine administration event 118 may include, without limitation, a recommended dosage of the medicine 102, a type of the medicine 102, and the schedule for administering the medicine 102. This recommendation is then transmitted back to the patient 104 in real time, which allows the patient 104 to adjust medicine 102 dosage, and more effectively treat the medical issue. The patient 104 provides feedback, which allows the iterative machine learning algorithm 110 to customize the medicine administration event 118 more closely to the needs of the patient 104.

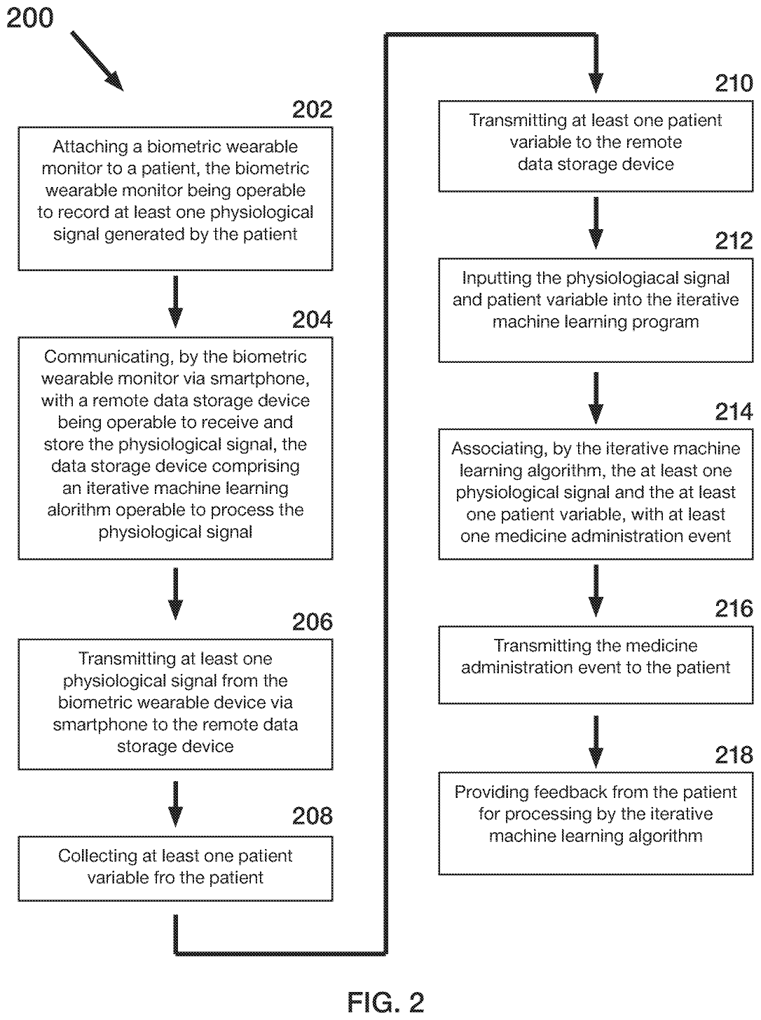

FIG. 2 references an exemplary computer-implemented method 200 for intelligent administering of medicine. The method 200 comprises an initial Step 202 of attaching a biometric wearable monitor to a patient, the biometric wearable monitor being operable to record at least one physiological signal generated by the patient. In an alternative embodiment, the method further comprises a step of recording, by the biometric wearable monitor, sleep and activity data of the patient.

Another Step 204 for method 200 may include communicating, by the biometric wearable monitor, with a remote data storage device, the data storage device being operable to receive and store the physiological signal, the data storage device comprising an iterative machine learning algorithm operable to process the physiological signal. Method 200 may further comprise a Step 206 of transmitting the at least one physiological signal from the biometric wearable monitor to the remote data storage device. The transmission of such datasets may be through radio frequency waves, Internet, and other communication means known in the art.

Continuing with method 200, a Step 208 includes collecting at least one patient variable from the patient. The patient variable may be collected on a written form, an online form, or a verbal question session. The patient variable may include: age, sex, current medication, current dosage of the medication, side effects, allergies, and medical history. In some embodiments, a Step 210 comprises transmitting the at least one patient variable to the remote data storage device. In this embodiment, the method 200 includes a mobile communication device, such as a smartphone, with a downloadable software application. The software application is defined by numerous functions for gathering outcome data from and transmitting medical cannabis or other medication dosing recommendations to the patient.

In some embodiments, a Step 212 includes inputting the physiological signal and patient variable into the iterative machine learning algorithm. Another Step 214 comprises associating, by the iterative machine learning algorithm, the at least one physiological signal and the at least one patient variable, with at least one medicine administration event. The iterative machine learning algorithm comprises a mathematical function operable to store and process a pre-processed dataset (physiological signals and patient variables). The pre-processed dataset is transmitted to the remote data storage device, and inputted into the iterative machine learning algorithm. After processing and model building with the dataset, the model is tested, and then the results are matched with an expected output, such as a medicine administration event, i.e., the recommended dosage of the medicine.

Method 200 may further comprise a Step 216 of transmitting the medicine administration event to the patient. The transmission may be to the mobile communication device carried by the patient. In another embodiment, method 200 further comprises a step of transmitting the medicine administration event to a mobile communication device of the patient. This data transmission step may also be in real time. The mobile communication device may include, without limitation, a smart phone, a tablet, a laptop, and a computer.

Method 200 also includes emitting, through the biometric wearable monitor or the mobile communication device, an alert to indicate reception of the medicine administration event. In yet another embodiment, the step of transmitting the medicine administration event to the patient may be at least partially based on the recorded sleep and activity data that was recorded by the biometric wearable monitor.

A final Step 218 comprises providing feedback from the patient for processing by the iterative machine learning algorithm. The patient feedback is required by the iterative machine learning algorithm to refine the recommendation, as each cycle of new data creates a dosage recommendation customized specifically for the patient. In alternative embodiments, method 200 involves repeating the transmission of the medicine administration event to a schedule. This data collection-dosage recommendation cycle is repeated using the medicine recommendations output of one cycle as the input for the next cycle. The cycles will repeat until the patient's medicine recommendations for this problem have stabilized at the optimal dosing for this problem in the patient. In machine learning terms, this is a gradient descent to an optimal point in the dosing space.

Although the process-flow diagrams show a specific order of executing the process steps, the order of executing the steps may be changed relative to the order shown in certain embodiments. Also, two or more blocks shown in succession may be executed concurrently or with partial concurrence in some embodiments. Certain steps may also be omitted from the process-flow diagrams for the sake of brevity. In some embodiments, some or all the process steps shown in the process-flow diagrams can be combined into a single process

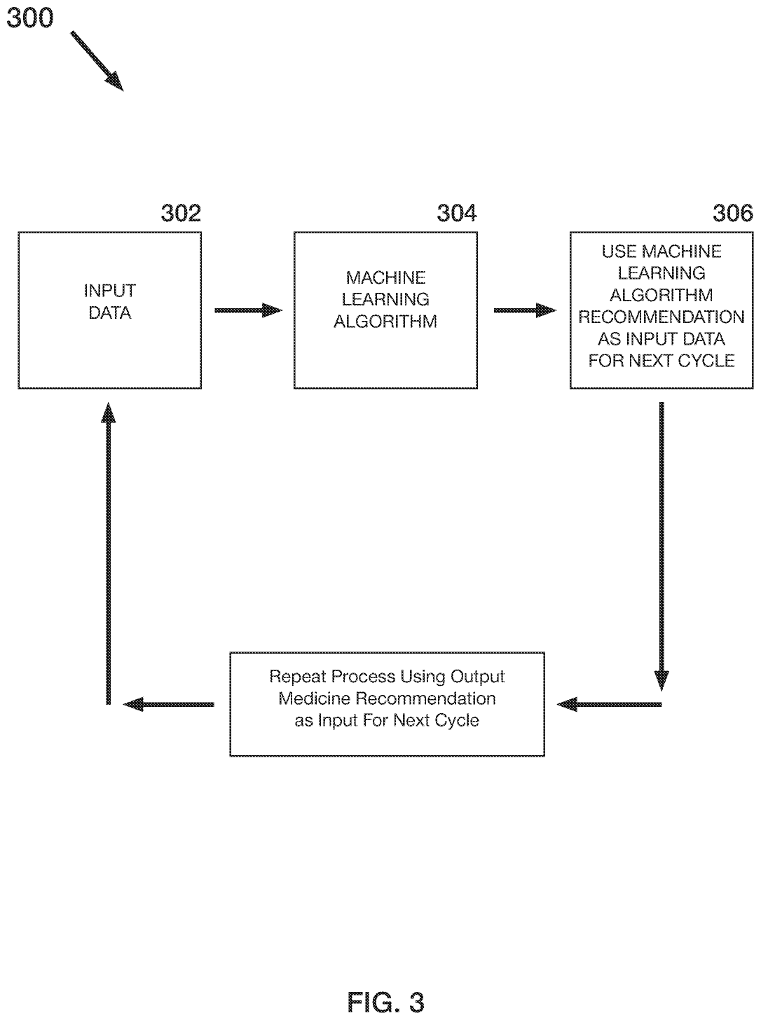

Turning now to the process chart 300 shown in FIG. 3, another embodiment of the method 300 utilizes the iterative machine learning algorithm to automate recommendations for an appropriate dosage of medication. In this embodiment, the method 300 also provide a mobile communication device, such as a smartphone with a downloadable software application.

In one embodiment, the software application includes numerous functions that are operable to gather the outcome data from and transmitting medication dosing recommendations to the patient. Also included in this embodiment, is a wearable activity monitor which records and transmits sleep and activity data to the smartphone software application; whereby the amount and type of medication is recommended at least partially based on the recorded activity.

Looking again at FIG. 3, steps for operating method 300 include a Step 302 of inputting data, chiefly from the wearable monitor device and the patient variable collected from the patient. The independent variables feeding the machine learning algorithm focuses on individual problems that a patient may have, e.g. pain, difficulty sleeping, anxiety, spasms. The set of independent variables contains different classes of input including:

problem inputs: specific problem with its attributes like diagnosis timeline;

patient inputs: e.g. age, sex, level of social support; dosing inputs: arrays: one for each medicine being used for this problem; and

outcome data (if available) on this dosing set: effects and side effects.

A subsequent Step 304 comprises providing a machine learning algorithm that processes the datasets to recommend optimal dosages for the patient. The machine learning algorithm is optimized to develop a new dosing set. As the algorithm gathers more inputs, it updates its recommendations for any one patient and also learns how to provide better recommendations for all patients. As the algorithm learns, the recommendations become more customized for the patient. A Step 306 is that the machine learning algorithm recommendation uses the inputted data for making recommendations in a subsequent cycle -iterating the data of the patient in the process. This is better than a random recommendation. The machine learning algorithm is configured to provide the machine learning algorithm recommendation. The machine learning algorithm recommendation may include a set of medicine recommendations for the patient, which have a high probability of generating a better outcome than the medicine dosages initially used for input into the machine learning algorithm.

A final Step requires iterative action by the machine learning algorithm to use one set of machine learning algorithm recommendations as part of the input data for the next cycle of collection and recommendation steps. This data collection-dosage recommendation cycle is repeated using the medicine recommendations output of one cycle as the input for the next cycle. The cycles will repeat until the patient's medicine recommendations for this problem have stabilized at the optimal dosing for this problem in the patient. In machine learning terms, this is a gradient descent to an optimal point in the dosing space.

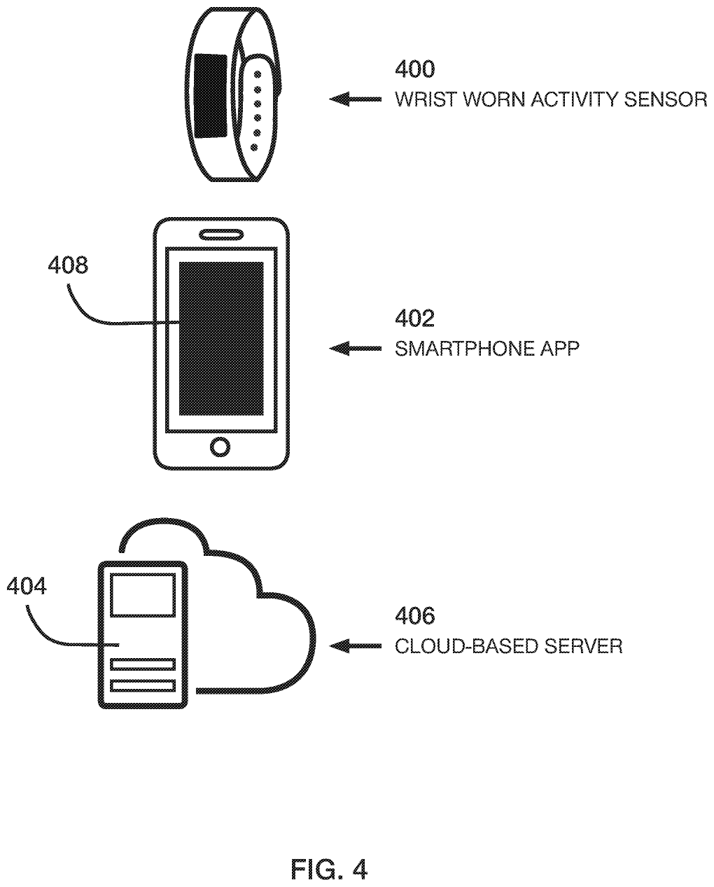

In yet another embodiment of system 100 and method 200, shown in block diagram 400 of FIG. 4, patient input data is collected when patient wears a biometric wearable monitor. In one non-limiting embodiment, a biometric wearable monitor is worn on the wrist during the initial phase of medicine recommending. Biometric wearable monitor is also worn as needed to optimize subsequent dosing recommendations. In one embodiment, biometric wearable monitor resembles a wristband activity sensor, and is operable to collect at least one physiological signal generated by the patient.

Biometric wearable monitor also serves to transmit two kinds of physiological signals to a software application on the patient mobile communication device. A first collected physiological signal generated by the patient may include sleep data: e.g. hours of sleep and sleep phases during the night. A second physiological signal may include steps taken by the patient, including number of steps taken by the patient during a day. These two classes of objective data will ultimately be used as independent variables to feed the machine learning algorithm.

In this configuration, a remote data storage device 404, i.e., cloud-based server 406, communicates with the smartphone 402 to receive the physiological signal from a biometric wearable monitor 400, and at least one patient variable directly from the patient. This is the functional element in the patient software application, which collects data sent from the activity sensor and transmits it to the remote data storage device, i.e., server. This functionality works in the background of the software application. This remote data storage device 404 may include a server communicating with the smartphone app. This server will collect activity data from the smartphone app. The remote data storage device transmits this activity data to the machine learning algorithm, which then converts the activity data into input for the machine learning algorithm to process.

In yet another embodiment, the mobile communication device of the patient includes a software application 408 that is downloadable on a smartphone 402. This creates a unique user interface for receiving recommendations for optimal medicine dosages. This configuration presents outcome data and recommendations to the user using an interface.

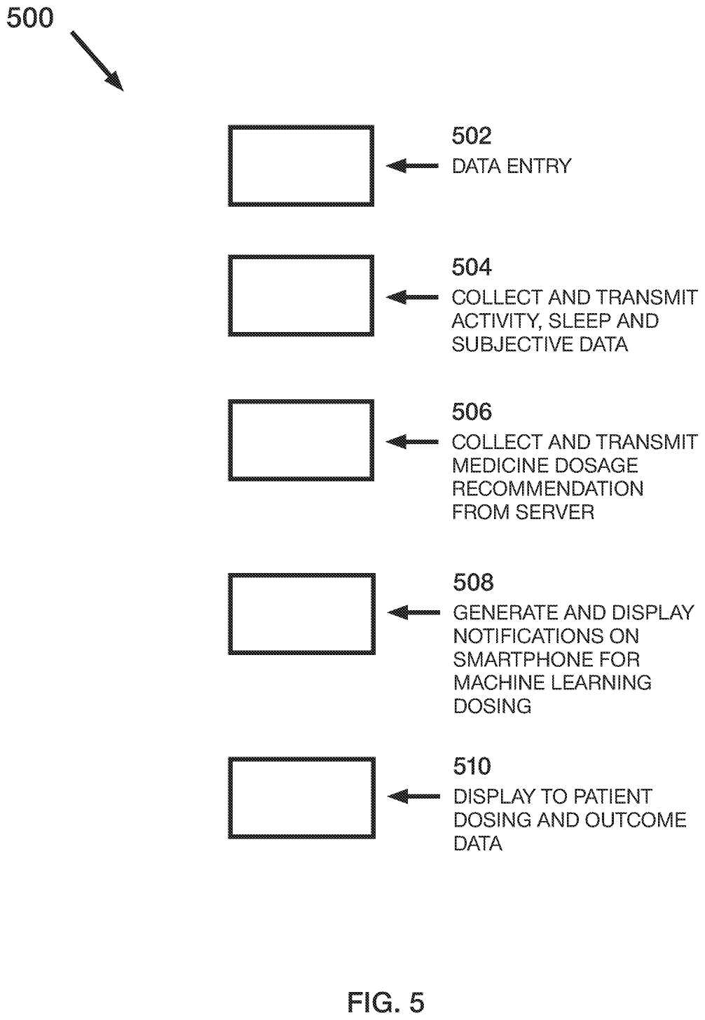

FIG. 5 shows alternative steps for a process 500 of automated recommendation of medicine. The process 500 initiates by data entry 502, which can involve signing in, collecting user profile, and the like. The process 500 then includes collecting and transmitting 504 activity data from the patient through the biometric wearable monitor. In one non-limiting embodiment, the software application on the mobile communication device is configured to collect data sent from the biometric wearable monitor, and transmit the data to the remote data storage device. The biometric wearable monitor is configured to receive sleep and steps activity data via Bluetooth from patient's activity sensor, and then transmit this data to the remote data storage device. This often occurs in the background.

In other embodiments, the process 500 may include collecting and transmitting 506 machine recommendations from the remote data storage device, i.e., server. The remote data storage device collects the data from the software application, and transmits the data to the iterative machine learning algorithm. This can also include collecting recommendations for medication (i.e. output from ML algorithm) from the server, and transmitting it to the patient. In a first embodiment, the transmission of medicine recommendations to the patient occurs via notifications on the smartphone.

Continuing, the process 500 also involves generating and displaying 508 notifications on a smartphone for the machine learning algorithm to process. The iterative machine learning algorithm converts the data into input for the iterative machine learning algorithm. This element contains scripts for generating the notifications to patient about medicine dosages. The process also includes displaying 510 medicine dosage and outcome data to the patient. This element contains software to display recent medicine dosing and outcome data to the patient on the smartphone.

In conclusion, system and method for intelligent administering of medicine recommends appropriate administration of a medicine through use of an iterative machine learning algorithm that learns from the behavior of a patient, through a biometric wearable monitor that collects physiological signals from sleeping and activity of the patient, and patient variables collected directly from the patient. Iterative machine learning algorithm adjusts the dosage of the medication based on different physiological signals, including: activity levels, sleeping patterns, emotional behavior, medical history, and medical needs.

After receiving the recommended dosage for the medicine, the patient provides feedback, which is inputted into the iterative machine learning algorithm. The feedback allows the iterative machine learning algorithm to customize the medicine administration event more closely to the needs of the patient.

In this manner, an appropriate dosage of medicine to the patient is based on patient feedback and on the sleep and activity behavior of the patient. This also works to increase the effectiveness of the medication by creating a more efficient and customizable dosage appropriate to the patient. This machine learning algorithm dramatically improves the results for the patient by maximizing the effectiveness of the medicine while reducing or even eliminating the potential side effects that result from inappropriate dosing.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A computer-readable storage device containing a set of instructions that causes a computer to perform a computer-implemented method for intelligent administering of medicine, the computer-implemented method comprising:
attaching a biometric wearable monitor to a patient, the biometric wearable monitor being operable to record at least one physiological signal generated by the patient;
communicating, by the biometric wearable monitor, with a remote data storage device, the data storage device being operable to receive and store the physiological signal, the data storage device comprising an iterative machine learning algorithm operable to process the physiological signal;
transmitting the at least one physiological signal from the biometric wearable monitor to the remote data storage device;
collecting at least one patient variable from the patient;
transmitting the at least one patient variable to the remote data storage device;
inputting the physiological signal and patient variable into the iterative machine learning algorithm;
associating, by the iterative machine learning algorithm, the at least one physiological signal and the at least one patient variable, with at least one medicine administration event;
transmitting the medicine administration event to the patient; and
providing feedback from the patient for processing by the iterative machine learning algorithm.

2. The method of claim 1, further comprising a step of attaching a biometric wearable monitor to at least one of the following body parts of the patient: the wrist, the legs, the ankle, the neck, the head, and the torso.

3. The method of claim 1, wherein the at least one physiological signal comprises an ECG signal and an accelerometer signal.

4. The method of claim 1, wherein the at least one medicine administration event includes at least one of the following: a dosage of the medicine, a type of the medicine, and the schedule for administering the medicine.

5. The method of claim 1, wherein the at least one patient variable includes at least one of the following: age, sex, current medication, current dosage of the medication, and side effects.

6. The method of claim 1, further comprising a step of transmitting the medicine administration event to a mobile communication device of the patient.

7. The method of claim 6, further comprising a step of emitting, through the biometric wearable monitor or the mobile communication device, an alert to indicate reception of the medicine administration event.

8. The method of claim 6, wherein the mobile communication device comprises a software application.

9. The method of claim 8, wherein the software application comprises a game-themed interface.

10. The method of claim 1, further comprising a step of recording, by the biometric wearable monitor, sleep and activity data of the patient.

11. The method of claim 10, further comprising a step of transmitting the medicine administration event to the patient, at least partially based on the recorded sleep and activity data.

12. The method of claim 1, further comprising a step of transmitting a recommended dosage of the medicine administration event to the patient in real time.

13. The method of claim 1, further comprising a step of repeating the transmission of the medicine administration event to a schedule.

14. The method of claim 1, wherein the remote data storage device includes at least one of the following: a server, a database, a cloud, and a processor.

15. The method of claim 1, wherein the medicine comprises medications, herbs, pills, syrups, IV's, Chinese medicine, and Western medicine.

16. A computer-readable storage device containing a set of instructions that causes a computer to perform a computer-implemented method for intelligent administering of medicine, the computer-implemented method comprising:
attaching a biometric wearable monitor to a patient, the biometric wearable monitor being operable to record at least one physiological signal generated by the patient;
communicating, by the biometric wearable monitor, with a remote data storage device, the data storage device being operable to receive and store the physiological signal, the data storage device comprising an iterative machine learning algorithm operable to process the physiological signal;
transmitting the at least one physiological signal from the biometric wearable monitor to the remote data storage device;
collecting at least one patient variable from the patient;
transmitting the at least one patient variable to the remote data storage device;
inputting the physiological signal and patient variable into the iterative machine learning algorithm;
associating, by the iterative machine learning algorithm, the at least one physiological signal and the at least one patient variable, with at least one medicine administration event;
transmitting the medicine administration event to the patient;
emitting, through the biometric wearable monitor or a mobile communication device, an alert to indicate reception of the medicine administration event, whereby the mobile communication device comprises a software application;
providing feedback from the patient for processing by the iterative machine learning algorithm; and
repeating the transmission of the medicine administration event to a schedule.

17. An automated system for intelligent administering of medicines, the system comprising:
a biometric wearable monitor detachably attachable to a patient having at least one patient variable;
at least one physiological signal generated by the patient and recorded by the biometric wearable monitor;
a remote data storage device being operable to receive and store the physiological signal and the patient variable collected from the patient;
an iterative machine learning algorithm integral in the remote data storage device, the iterative machine learning algorithm being operable to process the physiological signal and the patient variable, whereby the iterative machine learning algorithm associates at least one medicine administration event to the physiological signal and the patient variable; and
a mobile communication device having a software program, the software program receiving a transmission of the medicine administration event from the remote data storage device at a schedule.
